(19) United States
(12) Patent Application Publication
(10) Pub. No.: US 2015/0352275 A1
(43) Pub. Date: Dec. 10, 2015

(54) PORTABLE CONTACT-WASHING DEVICE FOR RELIEVING AND PREVENTING SYMPTOMS ASSOCIATED WITH HEMORRHOIDS

(71) Applicant: Yu LI, Johns Creek, GA (US)

(72) Inventor: Yu LI, Johns Creek, GA (US)

(21) Appl. No.: 14/760,480

(22) PCT Filed: Jan. 14, 2013
(86) PCT No.: PCT/US2013/021502

§ 371 Date: Jul. 13, 2015

Related U.S. Application Data
(60) Provisional application No. 61/586,836, filed on Jan. 15, 2012.

(51) Int. Cl. A61M 3/02

(52) U.S. Cl. CPC A61M 3/0279; A61M 3/0258; A61M 2205/586

(57) ABSTRACT

A portable contact-washing device allows a user to wear a single-use finger cover for contact washing of rectal area using water that is delivered to the finger pad area through micro tubing by a mini water pump. This contact wash uses a limited amount of water for a sufficient hygienic wash to the rectal area after a bowel movement without causing irritation which is often associated of using toilet paper. The contact washing process is efficient and requires a small volume of water, and the contact-washing device is highly portable and easy to use outside of home. The finger cover may be made of biodegradable material and will be discarded after the use.

### BACKGROUND

This invention relates to a portable washing device, specifically a portable contact-washing device for people, especially those with hemorrhoids symptoms to have an effective and flexible on-the-go method to achieve an adequate rectal hygiene.

A hemorrhoid (Piles) is a common adult ailment. National Institute of Health (NIH) estimated that by age 50, about half of adults have hemorrhoids, signals with the signs of itching, discomfort and bleeding after a bowl movement (http://www.nationalhemorrhoiddirectory.org). A separate study reported that 75 percent of people will have the symptom of hemorrhoids at some point in their lives (Baker H. Hemorrhoids. In: Longe J L, ed. Gale Encyclopedia of Medicine. 3rd ed. Detroit: Gale; 2006: 1766-1769.). Hemorrhoids is an ailment characterized by swollen and inflamed veins in ones anus and lower rectum; these hemorrhoidal symptoms may happen at any time due to excessive straining, rubbing, or cleaning around the anus, the irritation with bleeding and/or itching caused by inadequate hygiene may produce a vicious cycle of symptoms. The itching or pain is a sign of inflammation or a bodily reaction to allergy or cell-lining damage. Prolonged irritation and inflammation have been linked to other health problem, including obesity, heart disease and cancer.

The best way to prevent hemorrhoids is to keep stools soft so they pass easily, thus decreasing pressure and straining. A high fiber diet which may help soften stools often adds to the difficulty of cleaning the rectal area after bowel movement. Cleaning up after stool release is another essential part of hemorrhoid prevention. Wiping with toilet paper, which is traditionally used, provides inadequate hygiene, and repeated use of toilet paper leads to excess friction and damage to the skin membrane and cause bleeding. Excess usage of toilet paper is often required for the symptoms of constipation and diarrhea. The presence of hemorrhoids can make it even harder to clean the area immediately outside the rectum, yet good hygiene is essential to avoid irritation and promote healings.

A water wash is a better way to achieve adequate hygiene with minimal irritation. In addition to a regular shower, a bidet system or other similar washing systems provide adequate washing process to the rectal area. However people at works or at travel usually do not have the access to a washing device. A few portable washing devices were invented, including U.S. Pat. No. 4,259,954 and Patent No. US20080078847, which use portable washing bottles to spray water to the anus area for the cleaning. However, a portable water spray cannot provide an effective and hygienic wash. Due to its low efficiency, the water spay device has a larger size and is not easy to carry with.

### SUMMARY OF THE INVENTION

A portable contact-washing device allows a user to wear a single-use finger cover for contact washing of rectal area using water that is delivered to the finger pad area through micro tubing by a mini water pump. This contact wash uses a limited but consistent flow of water for any period of time to deliver a hygienic wash to the rectal area after a bowel movement without the irritation often caused from using toilet paper. The washing process is efficient and uses a small volume of water, and the device is highly portable and easy to use outside of home. The finger cover may be made of biodegradable material and will be discarded after the use.

### BRIEF DESCRIPTION OF THE DRAWINGS

FIG. 1 (A) is a perspective view of a contact-washing device with a mini water pump and a single-use finger cover, (B) is a front view of the single-use finger cover of (A), (C) is a perspective view of a variation of the single-use finger cover of (A), (D) is a front view of the single-use finger cover of (C).

FIG. 2 is a perspective view of a contact-washing device in a portable form of design: (A) the contact-washing device is in packed form for easy to carry with; (B) the contact-washing washing device is in the state of using with a single-use finger cover attached.

FIG. 3 is a perspective view of a contact-washing device in a wrist wearable design: a water container and a mini water pump are integrated together in a flat, wrist wearable design; the water pump has a short connection distance to the single-use finger cover.

FIG. 4 is a perspective view of a contact-washing device in which a squeezing water bag is used to provide water when the batteries are dead.

### DESCRIPTION OF PREFERRED EMBODIMENTS

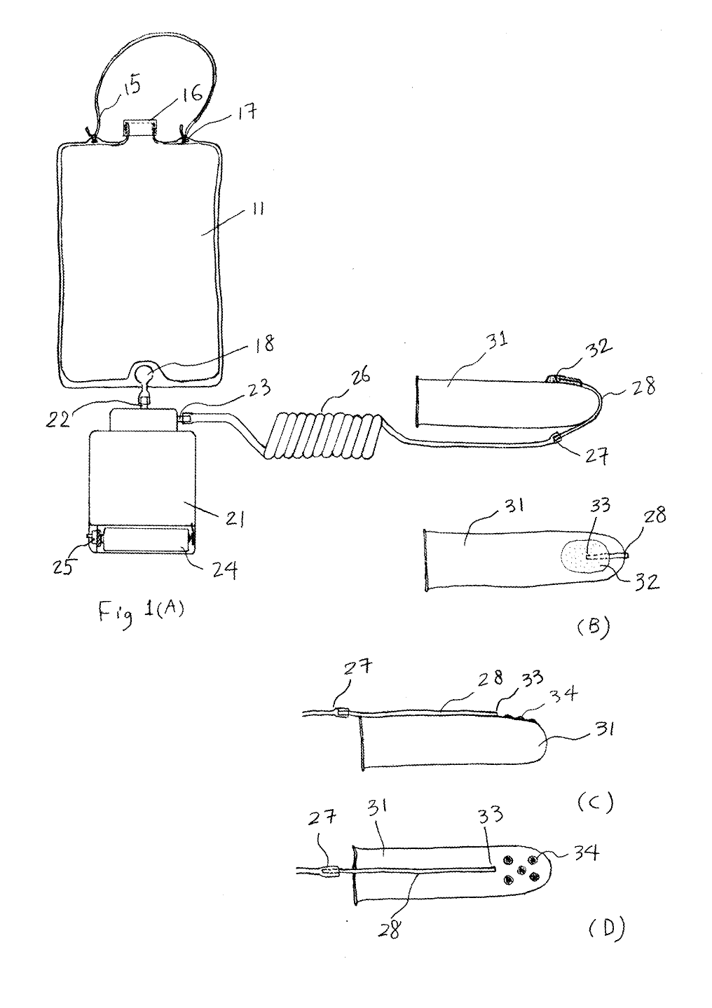

FIG. 1 is a perspective view of a contact-washing device constructed in accordance with the invention. A water bag 11 has a cap 16 and anchoring holes 17 of holding string 15. The string 15 is used to hold the water bag 11 on user's body or cloth. The bottom of the plastic bag 11 has an output tubing 18 which connect to the inlet 22 of a mini water pump 21, the water pump 21 is powered by a battery 24 and has its outlet 23 connect to a coiled tubing 26. Other end of tubing 26 connects to the micro tubing 28 through a micro tubing connector 27. Micro tubing 28 partially integrates with finger cover 31 and has an opening 33 near the finger pad area. An electric switch 25 is used to turn on or turn off the water flow.

FIG. 1 (A) shows a detailed view of finger cover 31 in one style. The finger cover 31 has a variable size to use, and is preferably used on the index or middle finger as those two fingers are more flexible and longer. At the front of the finger cover, a small, smooth, sponge pad 32 attaches to the finger pad area (FIG. 1 (B)). A micro tubing 28 reaches to the finger pad from the back of the finger cover and has openings inside of sponge pad 32, the other end of micro tubing 28 extends to micro tubing connector 27. The entire finger cover is made of bio-degradable materials and discarded into the toilet after the use.

FIG. 1(C, D) shows a finger cover in a different style: no sponge pad is used, the micro tubing integrates with the finger cover at the front to reach the finger pad area; the finger pad area has round bumps 34 to improve the washing efficiency.

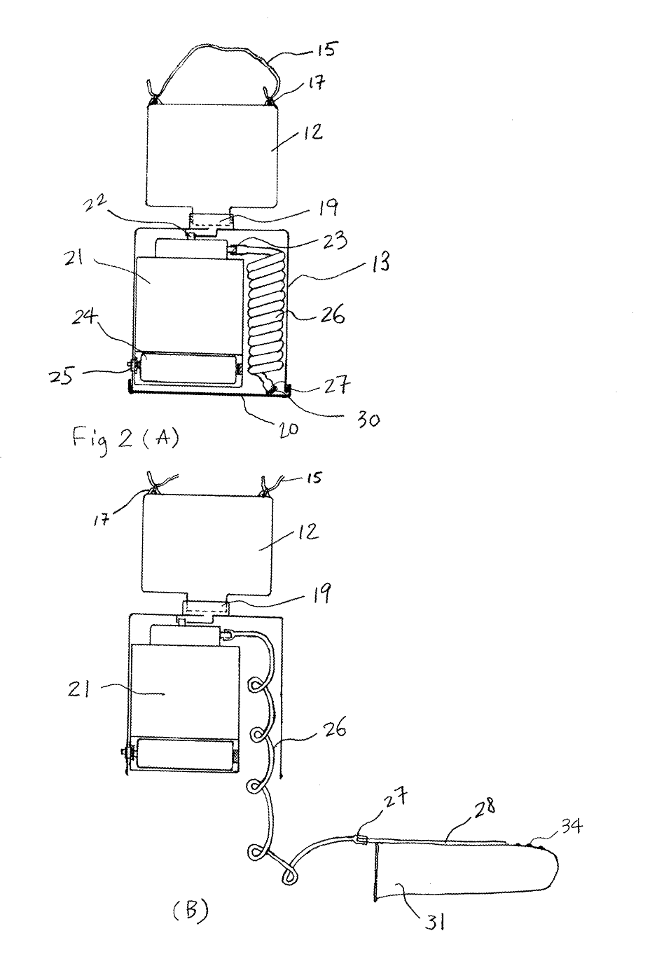

FIG. 2 shows a portable water pump device in which the coiled tubing 26 and water pump 21 are made to be inside a portable container 13 when the device is not in use (FIG. 2 (A)). A cover 20 seals the container 13. A screw top 19 of Mini pump 21 connects to the screw top of the water container 12 directly with inlet 22 opening to water container. Micro tubing connector 27 has a small cover 30 at the end to prevent water dripping. FIG. 2 (B) shows the device is in the form of using: the coiled tubing 26 is pulled out to allow the finger cover reaching a washing area. Coiled tubing 26 contracts automatically to its original position after release from finger cover 31.

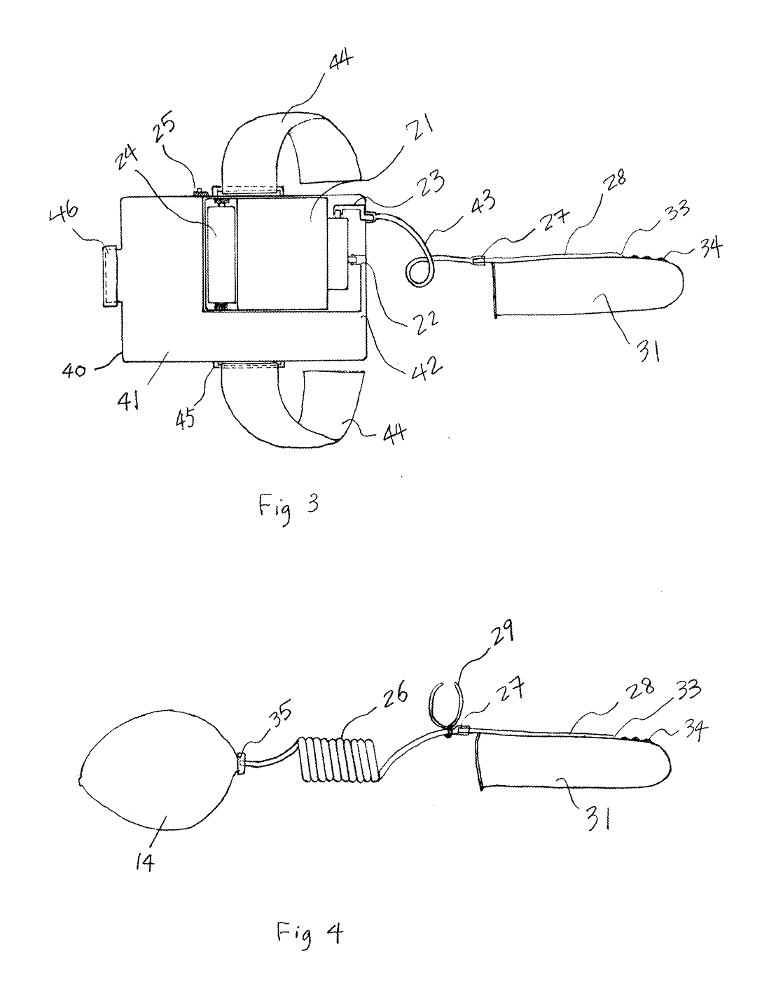

FIG. 3 shows an integrated water pump device in which water pump 21 and battery 24 were assembled inside of a flat shape water container 40, water is filled to the compartment 41 which is closed with cap 46, container 40 is wrapped at a user's wrist using bands 44. Bands 44 attach to container 40 at the anchors 45. The water opens to the water pump inlet 22 directly at 42. Container 40 close to user's hand, a shorter tubing 43 connect the water pump to the finger cover 31.

FIG. 4 is a detailed view of a squeezable water bag 14 which can be used as an alternate to the water pump device of FIG. 1. A squeezable water bag is smaller and can be hold by hand; it is highly portable and can be used when the battery powered device (FIG. 1) is not available or the batteries are dead. A thumb ring 29 holds tubing 26 close to a user's hand. A cap 35 closes the water bag with the coiled tubing connected.

### REFERENCE NUMERALS

11 foldable water bag, 12 water container, 13 water pump container, 14 squeezable water bag, 15 strings, 16 water bag cap, 17 water bag string anchor, 18 water bag output tubing, 19 connecting screw ring, 20 container cover, 21 mini water pump, 22 water pump inlet, 23 water pump outlet, 24 batteries, 25 water pump switch, 26 coiled flexible tubing, 27 micro tubing connector, 28 soft micro tubing, 29 thumb ring, 30 micro tubing connector cover, 31 single-use finger cover, 32 sponge pad, 33 micro tubing opening, 34 mini round bumps, 35 cap, 40 a wrist wearable unit, 41 water filling space, 42 opening connects to pump inlet, 43 flexible tubing, 44 device holding bands, 45 bands anchors, 46 cap.

### OPERATION

In operation: a user fills water bag 11 (FIG. 1) with water, closes cap 16, and hangs water bag 11 around own neck using string 15; then put a single-use finger cover 31 on own index finger or middle finger and connects micro tubing 28 to micro tubing connector 27. When it is ready for a wash, the user turns on the switch 25, and uses finger pad to wash the rectal area with water delivered constantly to the finger pad area at the opening 33 of micro tubing 28. When performing the washing, the user's hand and the finger with finger cover points downward, a slow flow of water from micro tubing 28 make little splash; the washing water drips to the toilet, and user's hand and other fingers are kept clean and dry. After the wash, the user turns off the power switch, and removes finger cover 31 by detaching the micro tubing 28 from micro tubing connector 27 and pull off used finger cover 31 from the finger with the help of the other hand. The used finger cover will be discarded into a trash or a toilet. The plastic water bag, mini pump and coiled tubing are folded and stored away for future use. A normal wash takes 10-20 seconds with a water flow rate of 50-100 ml per minutes; a volume of 50 ml of water provides an adequate wash. A user may choose to take a longer wash by using a large size water bag or refill the regular water bag.

FIG. 2 shows a portable style of the contact-washing device: the coiled tubing 26 and water pump 21 are made to fit into a single portable container for easy carry and use. The coiled tubing 26 is made of a material that has a memory of its original position. The coiled tubing is stretched from its original position into fully extended length to reach a washing area for a contact wash, and is released after the use to returns to its original position. The use of the finger cover 31 is similar to the description above. The water container directly screws to the container 13. A different size of water containers can be used with the water pump unit for an extended period of wash.

FIG. 3 shows a portable style of the contact-washing device to be worn on user's wrist. After filled with water, the flat shape contact-washing device is worn at user's wrist with bands 44. The use of the finger cover 31 is similar to the description above. The tubing 26 is shorter and does not need a coiled form.

FIG. 4 shows a contact-washing device using a squeezable water bag 14 instead of a water pump 21. The use of the finger cover 31 is similar to the description above, but the water is delivered by squeezing water bag 14 for the washing process. This manual contact washing device provides a similar washing results but is more difficult to control the flow of water in comparison with electric unit.

I claim:

1. A portable contact-washing device comprising, in combination,
a. a single-use finger cover integrated with a soft micro tubing wherein one end of said micro tubing has an opening near the finger pad area of said finger cover, and other end of said micro tubing connects detachably to a mini water pump through a micro tubing connector.
b. a water container connects to said mini electric water pump and said finger cover through a coiled tubing and said micro tubing connector wherein a water stream is delivered to said finger pad area through said coiled tubing and said micro tubing when said water pump is turned on.

2. The finger cover of claim 1 wherein said finger cover is made of thin plastic or rubber materials and covers entire a finger.

3. The finger cover of claim 1 wherein a sponge pad is added to said finger pad area and covers said micro tubing opening.

4. The finger cover of claim 1 wherein said finger pad has round bumps in front of said micro tubing opening.

5. The mini electric water pump of claim 1 has a switch to turn on or turn off of water flow.

6. The contact-washing device of claim 1 wherein said water pump and said coiled tubing are installed into one portable compartment unit and said coiled tubing is pulled out to extend said finger cover to a washing area.

7. The coiled tubing of claim 1 wherein said coiled tubing is made of plastics with memory of its original position.

8. The coiled tubing of claim 1 wherein said coiled tubing winds or unwinds through a spring wheel.

9. The coiled tubing of claim 1 wherein said coiled tubing has a thumb loop near said micro tubing connector to hold said coiled tubing to a user's hand.

10. The contact-washing device of claim 1 wherein said mini electric water pump and said water container are made into a wrist wearable shape and size.

11. The contact-washing device of claim 1 wherein said water pump is replaceable by a squeezable water container to provide water to finger cover through said coiled tubing and said micro tubing.

12. The single-use finger cover of claim 1 wherein said finger cover is made of bio-degradable materials.

13. A method of using said contact-washing device with said single-use finger cover for rectal area washing after a bowl movement, comprising:
a. a user wear said contact-washing device and control the water flow to said finger cover,
b. A user wear said finger cover on a finger to reach to the rectal area for a contact wash using said finger pad,
c. Water flows to said finger pad area through micro tubing controlled by said water pump for variable wash speed and time;
d. After a wash, said finger cover is detached from said micro tubing connector, removed from finger, and discarded.
e. After a wash, said coiled tubing retracted to its original position for cleanness and to be easy to carry with.

14. A method of using said contact-washing device of claim 13 wherein said contact-washing device is used for a wash of other contaminated or affected area.
